(19) United States
(12) Patent Application Publication
(10) Pub. No.: US 2003/0051988 A1
(43) Pub. Date: Mar. 20, 2003

(54) Treatment of crude oil fractions, fossil fuels, and products thereof with ultrasound

(76) Inventors: Rudolf W. Gunnerman, Reno, NV (US); Paul S. Moote, Park City, UT (US); Mark T. Cullen, Reno, NV (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/863,127

(22) Filed: May 22, 2001

(51) Int. Cl. C07C 1/00; C07C 2/00; C07C 4/00; C07C 5/00

(52) U.S. Cl. 204/157.15; 204/157.6; 204/157.78; 44/904

(57) ABSTRACT

Crude oil fractions, fossil fuels, and organic liquids in general in which it is desirable to reduce the levels of fused-ring compounds, aromatics, and olefins are combined with an aqueous phase to form an emulsion and treated with ultrasound with the effect of opening rings in the fused-ring compounds, saturating aromatics, and converting the olefins to paraffins. In fossil fuels and crude oil fractions, the process raises the API gravity, and in diesel fuels, the process raises the cetane index and thereby improves performance. In fuels and fractions with sulfur-containing and nitrogen-containing components, the process reduces the level of these compounds. The process can be performed both with and without the added presence of hydrogen peroxide. The invention is performed either as a continuous process or a batch process.

### BACKGROUND OF THE INVENTION

1. Field of the Invention

This invention resides in the field of chemical processes for the treatment of crude oil fractions and the various types of products derived and obtained from these sources. In particular, this invention addresses reformation processes as ring-opening reactions and the saturation of double bonds, to upgrade fossil fuels and convert organic products to forms that will improve their performance and expand their utility. This invention also resides in the removal of sulfur-containing compounds, nitrogen-containing compounds, and other undesirable components from petroleum and petroleum-based fuels.

2. Description of the Prior Art

Fossil fuels are the largest and most widely used source of power in the world, offering high efficiency, proven performance, and relatively low prices. There are many different types of fossil fuels, ranging from petroleum fractions to coal, tar sands, and shale oil, with uses ranging from consumer uses such as automotive engines and home heating to commercial uses such as boilers, furnaces, smelting units, and power plants.

Fossil fuels and other crude oil fractions and products derived from natural sources contain a vast array of hydrocarbons differing widely in molecular weight, boiling and melting points, reactivity, and ease of processing. Many industrial processes have been developed to upgrade these materials by removing, diluting, or converting the heavier components or those that tend to polymerize or otherwise solidify, notably the olefins, aromatics, and fused-ring compounds such as naphthalenes, indanes and indenes, anthracenes, and phenanthracenes. A common means of effecting the conversion of these compounds is saturation by hydrogenation across double bonds.

For fossil fuels in particular, a growing concern is the need to remove sulfur compounds. Sulfur from sulfur compounds causes corrosion in pipeline, pumping, and refining equipment, the poisoning of catalysts used in the refining and combustion of fossil fuels, and the premature failure of combustion engines. Sulfur poisons the catalytic converters used in diesel-powered trucks and buses to control the emissions of oxides of nitrogen (NOₓ). Sulfur also causes an increase in particulate (soot) emissions from trucks and buses by degrading the soot traps used on these vehicles. The burning of sulfur-containing fuel produces sulfur dioxide which enters the atmosphere as acid rain, inflicting harm on agriculture and wildlife, and causing hazards to human health.

The Clean Air Act of 1964 and its various amendments have imposed sulfur emission standards that are difficult and expensive to meet. Pursuant to the Act, the United States Environmental Protection Agency has set an upper limit of 15 parts per million by weight (ppmw) on the sulfur content of diesel fuel, effective in mid-2006. This is a severe reduction from the standard of 500 ppmw in effect in the year 2000. For reformulated gasoline, the standard of 300 ppmw in the year 2000 has been lowered to 30 ppmw, effective Jan. 1, 2004. Similar changes have been enacted in the European Union, which will enforce a limit of 50 ppmw sulfur for both gasoline and diesel fuel in the year 2005. The treatment of fuels to achieve sulfur emissions low enough to meet these requirements is difficult and expensive, and the increase in fuel prices that this causes will have a major influence on the world economy.

The principal method of fossil fuel desulfurization in the prior art is hydrodesulfurization, i.e., the reaction between the fossil fuel and hydrogen gas at elevated temperature and pressure in the presence of a catalyst. This causes the reduction of organic sulfur to gaseous H₂S, which is then oxidized to elemental sulfur by the Claus process. A considerable amount of unreacted H₂S remains however, with its attendant health hazards. A further limitation of hydrodesulfurization is that it is not equally effective in removing all sulfur-bearing compounds. Mercaptans, thioethers, and disulfides, for example, are easily broken down and removed by the process, while aromatic sulfur compounds, cyclic sulfur compounds, and condensed multicyclic sulfur compounds are less responsive to the process. Thiophene, benzothiophene, dibenzothiophene, other condensed-ring thiophenes, and substituted versions of these compounds, which account for as much as 40% of the total sulfur content of crude oils from the Middle East and 70% of the sulfur content of West Texas crude oil, are particularly refractory to hydrodesulfurization.

In addition to sulfur-bearing compounds, nitrogen-bearing compounds are also sought to be removed from fossil fuels, since these compounds tend to poison the acidic components of the hydrocracking catalysts used in the refinery. The removal of nitrogen-bearing compounds is achieved by hydrodenitrogenation, which is a hydrogen treatment performed in the presence of metal sulfide catalysts. Both hydrodesulfurization and hydrodenitrogenation require expensive catalysts as well as high temperatures (typically 400° F. to 850° F., which is equivalent to 204° C. to 254° C.) and pressures (typically 50 psi to 3,500 psi). These processes require a source of hydrogen or an on-site hydrogen production unit, which entails high capital expenditures and operating costs. In both of these processes, there is also a risk of hydrogen leaking from the reactor.

Of possible relevance to this invention are co-pending U.S. patent application Ser. No. 09/676,260, entitled “Oxidative Desulfurization of Fossil Fuels With Ultrasound,” Teh Fu Yen, et al., inventors, filed Sep. 28, 2000, and co-pending U.S. patent application Ser. No. 09/812,390, entitled “Continuous Process for Oxidative Desulfurization of Fossil Fuels With Ultrasound and Products Thereof,” Rudolf W. Gunnerman, inventor, filed Mar. 29, 2001. Both of these applications are incorporated herein by reference in their entirety for all legal purposes capable of being served thereby.

### SUMMARY OF THE INVENTION

It has now been discovered that fossil fuels, crude oil fractions, and many of the components that are derived from these sources can undergo a variety of beneficial conversions and be upgraded in a variety of ways by a process that applies ultrasound to these materials in a multiphase reaction medium. The organic material is combined with an aqueous phase to form an emulsion, placing the phases in intimate contact during the exposure to ultrasound. Hydrogen gas is not required, nor are the high temperature and pressure that are commonly needed for hydrogenations of the prior art. In certain embodiments of the invention, the ultrasound treatment is performed in the presence of a hydroperoxide, and in certain embodiments as well, a transition metal catalyst is used. One of the surprising discoveries associated with certain embodiments of this invention, however, is that the conversions achieved by this invention can be achieved without the inclusion of a hydroperoxide in the reaction mixture.

Included among the conversions achieved by the present invention are the removal of organic sulfur compounds, the removal of organic nitrogen compounds, the saturation of double bonds and aromatic rings, and the opening of rings in fused-ring structures. The API gravities of fossil fuels and crude oil fractions are raised (i.e., the densities lowered) as a result of treatments in accordance with the invention. The invention thus resides in part in the process of using ultrasound to achieve these conversions. The invention further resides in processes for converting aromatics to cycloparaffins, and opening one or more rings in a fused-ring structure, thereby for example converting naphthalenes to monocyclic aromatics, anthracenes to naphthalenes, fused heterocyclic rings such as benzothiophenes, dibenzothiophenes, benzofurans, quinolines, indoles, and the like to substituted benzenes, acenaphthalenes and acenaphthenes to indanes and indenes, and monocyclic aromatics to noncyclic structures. Further still, the invention resides in processes for converting olefins to paraffins, and in processes for breaking carbon-carbon bonds, carbon-sulfur bonds, carbon-metal bonds, and carbon-nitrogen bonds.

In addition to raising the API gravity of a petroleum-based fuel, the invention raises the cetane index of petroleum fractions and cracking products whose boiling points or ranges are in the diesel range. The term “diesel range” is used herein in the industry sense to denote the portion of crude oil that distills out after naphtha, and generally within the temperature range of approximately 200° C. (392° F.) to 370° C.(698° F.). Fractions and cracking products whose boiling ranges are contained in this range as well as those that overlap with this range to a majority extent are included. Examples of refinery fractions and streams within the diesel range are fluid catalytic cracking (FCC) cycle oil fractions, coker distillate fractions, straight run diesel fractions, and blends. The invention also imparts other beneficial changes such as a lowering of boiling points and a removal of components that are detrimental to the performance of the fuel and those that affect refinery processes and increase the cost of production of the fuel. Thus, for example, FCC cycle oils can be treated in accordance with the invention to sharply reduce their aromatics content.

A further group of crude oil fractions for which the invention is particularly useful are gas oils, which term is used herein as it is in the petroleum industry, to denote liquid petroleum distillates that have higher boiling points than naphtha. The initial boiling point may be as low as 400° F. (200° C.), but the preferred boiling range is about 500° F. to about 1100° F. (approximately equal to 260° C. to 595° C.). Examples of fractions boiling within this range are FCC slurry oil, light and heavy gas oils, so termed in view of their different boiling points, and coker gas oils. All of the terms in this and the preceding paragraph are used herein as they are in the petroleum art.

By virtue of the conversions that occur as a result of the process of this invention, hydrocarbon streams experience changes in their cold flow properties, including their pour points, cloud points, and freezing points. Sulfur compounds, nitrogen compounds, and metal-containing compounds are also reduced, and the use of a process in accordance with this invention significantly lessens the burden on conventional processes such as hydrodesulfurization, hydrodenitrogenation, and hydrodemetallization, which can therefore be performed with greater effectiveness and efficiency.

These and other advantages, features, applications and embodiments of the invention are made more apparent by the description that follows.

### DETAILED DESCRIPTION OF THE INVENTION AND SPECIFIC EMBODIMENTS

The term “liquid fossil fuel” is used herein to denote any carbonaceous liquid that is derived from petroleum, coal, or any other naturally occurring material, as well as processed fuels such as gas oils and products of fluid catalytic cracking units, hydrocracking units, thermal cracking units, and cokers, and that is used to generate energy for any kind of use, including industrial uses, commercial uses, governmental uses, and consumer uses. Included among these fuels are automotive fuels such as gasoline, diesel fuel, jet fuel, and rocket fuel, as well as petroleum residuum-based fuel oils including bunker fuels and residual fuels. No. 6 fuel oil, for example, which is also known as “Bunker C” fuel oil, is used in oil-fired power plants as the major fuel and is also used as a main propulsion fuel in deep draft vessels in the shipping industry. No. 4 fuel oil and No. 5 fuel oil are used to heat large buildings such as schools, apartment buildings, and office buildings, and large stationary marine engines. The heaviest fuel oil is the vacuum residuum from the fractional distillation, commonly referred to as “vacuum resid,” with a boiling point of 565° C. and above, which is used as asphalt and coker feed. The present invention is useful in the treatment of any of these fuels and fuel oils for purposes of reducing the sulfur content, the nitrogen content, and the aromatics content, and for general upgrading to improve performance and enhance utility. Certain embodiments of the invention involve the treatment of fractions or products in the diesel range which include, but are not limited to, straight-run diesel fuel, feed-rack diesel fuel (as commercially available to consumers at gasoline stations), light cycle oil, and blends of straight-run diesel and light cycle oil ranging in proportion from 10:90 to 90:10 (straight-run diesel:light cycle oil).

The term “crude oil fraction” is used herein to denote any of the various refinery products produced from crude oil, either by atmospheric distillation or vacuum distillation, including fractions that have been treated by hydrocracking, catalytic cracking, thermal cracking, or coking, and those that have been desulfurized. Examples are light straight-run naphtha, heavy straight-run naphtha, light steam-cracked naphtha, light thermally cracked naphtha, light catalytically cracked naphtha, heavy thermally cracked naphtha, reformed naphtha, alkylate naphtha, kerosene, hydrotreated kerosene, gasoline and light straight-run gasoline, straight-run diesel, atmospheric gas oil, light vacuum gas oil, heavy vacuum gas oil, residuum, vacuum residuum, light coker gasoline, coker distillate, FCC (fluid catalytic cracker) cycle oil, and FCC slurry oil.

The term “fused-ring aromatic compound” is used herein to denote compounds containing two or more fused rings at least one of which is a phenyl ring, with or without substituents, and including compounds in which all fused rings are phenyl or hydrocarbyl rings as well as compounds in which one or more of the fused rings are heterocyclic rings. Examples are substituted and unsubstituted naphthalenes, anthracenes, benzothiophenes, dibenzothiophenes, benzofurans, quinolines, and indoles.

The term “olefins” is used herein to denote hydrocarbons, primarily those containing two or more carbon atoms and one or more double bonds.

Fossil fuels and crude oil fractions treated by ultrasound in accordance with this invention have significantly improved properties relative to the same materials prior to treatment, these improvements rendering the products unique and improving their usefulness as fuels. One of these properties is the API gravity. The term “API gravity” is used herein as it is among those skilled in the art of petroleum and petroleum-derived fuels. In general, the term represents a scale of measurement adopted by the American Petroleum Institute, the values on the scale increasing as specific gravity values decrease. Thus, a relatively high API gravity means a relatively low density. The API gravity scale extends from −20.0 (equivalent to a specific gravity of 1.2691) to 100.0 (equivalent to a specific gravity of 0.6112).

To illustrate the range of applicability of the present invention and its effect on liquid fossil fuels and crude oil fractions, Table I below lists unit streams within the diesel boiling range and unit streams within the gas oil boiling range, with a range of values for various parameters for stream, and a range of how much each parameter can be reduced or increased by the ultrasound treatment of the present invention.

TABLE I
Refinery Unit Streams: Parameter Ranges and Effects of Invention:
(+) Denotes Increase; (−) Denotes Reduction
Diesel Boiling Range Materials
(Boiling Range 300-750° F., 150-400° C.):

| | FCC Cycle | Crude Straight- | |
|---|---|---|---|
| Unit Stream | Oil | Run Diesels | Coker Distillates |
| API | 15-30 | 27-45 | 27-45 |
| change | (+) 3-30 | (+) 1-15 | (+) 0-15 |
| Cetane index | 25-40 | 30-60 | 28-40 |
| change | (+) 1-30 | (+) 1-20 | (+) 1-25 |
| Aromatics (weight %) | 30-95 | 0-50 | 30-95 |
| change | (−) 1-(−) 50 | (−) 1-(−) 25 | (−) 1-(−) 40 |
| Nitrogen (ppm as N) | 0-10,000 | 0-10,000 | 0-10,000 |
| change (as % of initial | (−) 1-90 | (−) 1-90 | (−) 1-90 |
| value) | | | |
| Bromine number | 5-40 | 0-5 | 10-50 |
| change (as %) | (−) 1-95 | (−) 1-95 | (−) 1-95 |
| Sulfur (weight % as S) | 0.05-5 | 0.01-5 | 0.05-5 |
| change (as % of initial | (−) 5-95 | (−) 5-95 | (−) 5-95 |
| value) | | | |
| Pour Point (° F.) | 10-40 | (−10)-40 | 0-30 |
| change (° F.) | (−) 5-50 | (−) 5-50 | (−) 5-50 |
| Cloud Point (° F.) | 10-45 | (−10)-45 | 0-35 |
| change (° F.) | (−) 5-50 | (−) 5-50 | (−) 5-50 |

Gas Oil Boiling Range Materials
(Boiling Range 500-1100° F., 260-595° C.):

| | FCC Cycle | | |
|---|---|---|---|
| Unit Stream → | Oil | Crude Gas oils | Coker Gas oils |
| API | (−15)-25 | 5-35 | 0-30 |
| change | (+) 1-40 | (+) 1-25 | (+) 1-30 |
| Aromatics (weight %) | 30-95 | 0-50 | 20-80 |
| change | (−) 1-(−) 80 | (−) 1-(−) 40 | (−) 1-(−) 50 |
| Nitrogen (ppm as N) | 0-10,000 | 0-10,000 | 0-10,000 |
| change (as % of initial | (−) 1-90 | (−) 1-90 | (−) 1-90 |
| value) | | | |
| Change Carbon | (−) 1-90 | (−) 1-90 | (−) 1-90 |
| Residue | | | |
| (Conradson) (%) | | | |
| Sulfur (weight % as S) | 0.05-7 | 0.01-7 | 0.05-7 |
| change (as % of initial | (−) 5-95 | (−) 5-95 | (−) 5-95 |
| value) | | | |
| Pour Point (° F.) | 50-150 | 50-150 | 50-150 |
| change (° F.) | (−) 5-100 | (−) 5-100 | (−) 5-100 |

The ultrasound process of the present invention is applicable to any liquid fossil fuels, preferably those with API gravities within the range of −10 to 50, and most preferably within the range of 0 to 45. For materials boiling in the diesel range, the process of the invention is preferably performed in such a manner that the starting materials are converted to products with API gravities within the range of 37.5 to 45. FCC cycle oils are preferably converted to products with API gravities within the range of 30 to 50. For liquid fossil fuels in general, the process of the invention is preferably performed to achieve an increase in API gravity by an amount ranging from 2 to 30 API gravity units, and more preferably by an amount ranging from 7 to 25 units. Alternatively stated, the invention preferably increases the API gravity from below 20 to above 35.

As stated above, fossil fuels boiling within the diesel range that are treated in accordance with this invention experience an improvement in their cetane index (also referred to in the art as the “cetane number”) upon being treated in accordance with this invention. Diesel fuels to which the invention is of particular interest in this regard are those having a cetane index greater than 40, preferably within the range of 45 to 75, and most preferably within the range of 50 to 65. The improvement in cetane index can also be expressed in terms of an increase over that of the material prior to ultrasound treatment. In certain preferred embodiments, the increase is by an amount ranging from 1 to 40 cetane index units, and more preferably by an amount ranging from 4 to 20 units. As a still further means of expression, the invention preferably increases the cetane index from below 47 to about 50. This invention can be used to produce diesel fuels having a cetane index of greater than 50.0, or preferably greater than 60.0. In terms of ranges, the invention is capable of producing diesel fuels having a cetane index of from about 50.0 to about 80.0, and preferably from about 60.0 to about 70.0. The cetane index or number has the same meaning in this specification and the appended claims that it has among those skilled in the art of automotive fuels.

As noted above, certain embodiments of the invention involve the inclusion of a hydroperoxide in the reaction mixture. The term “hydroperoxide” is used herein to denote a compound of the molecular structure

R—O—O—H

in which R represents either a hydrogen atom or an organic or inorganic group. Examples of hydroperoxides in which R is an organic group are water-soluble hydroperoxides such as methyl hydroperoxide, ethyl hydroperoxide, isopropyl hydroperoxide, n-butyl hydroperoxide, sec-butyl hydroperoxide, tert-butyl hydroperoxide, 2-methoxy-2-propyl hydroperoxide, tert-amyl hydroperoxide, and cyclohexyl hydroperoxide. Examples of hydroperoxides in which R is an inorganic group are peroxonitrous acid, peroxophosphoric acid, and peroxosulfuric acid. Preferred hydroperoxides are hydrogen peroxide (in which R is a hydrogen atom) and tertiary-alkyl peroxides, notably tert-butyl peroxide.

The aqueous fluid that is combined with the fossil fuel or other liquid organic starting material in the processes of this invention may be water or any aqueous solution. The relative amounts of organic and aqueous phases may vary, and although they may affect the efficiency of the process or the ease of handling the fluids, the relative amounts are not critical to this invention. In most cases, however, best results will be achieved when the volume ratio of organic phase to aqueous phase is from about 8:1 to about 1:5, preferably from about 5:1 to about 1:1, and most preferably from about 4:1 to about 2:1.

When a hydroperoxide is present, the amount of hydroperoxide relative to the organic and aqueous phases can also be varied, and although the conversion rate and yield may vary somewhat with the proportion of hydroperoxide, the actual proportion is not critical to the invention, and any excess amounts will be eliminated by the ultrasound. When the hydroperoxide is added as a solute in the aqueous solution that is used as the aqueous phase, best results are generally achieved with a hydroperoxide concentration of from about 10 ppm to about 100 ppm by weight, and preferably from about 15 ppm to about 50 ppm by weight, of the aqueous solution, particularly when the hydroperoxide is H₂O₂. Alternatively, when the H₂O₂ amount is calculated as a component of the combined organic and aqueous phases, best results will generally be achieved in most systems with an H₂O₂ concentration within the range of from about 0.0003% to about 0.03% by volume (as H₂O₂), and preferably from about 0.001% to about 0.01%, of the combined phases. For hydroperoxides other than H₂O₂, the preferred concentrations will be those of equivalent molar amounts.

In certain embodiments of this invention as well, a surface active agent or other emulsion stabilizer is included to stabilize the emulsion as the organic and aqueous phases are being prepared for the ultrasound exposure. Certain petroleum fractions contain surface active agents as naturally-occurring components of the fractions, and these agents may serve by themselves to stabilize the emulsion. In other cases, synthetic or non-naturally-occurring surface active agents can be added. Any of the wide variety of known materials that are effective as emulsion stablizers can be used. Listings of these materials are available McCutcheon's Volume 1: Emulsifiers & Detergents—1999 North American Edition, McCutcheon's Division, MC Publishing Co., Glen Rock, N.J., USA, and other published literature. Cationic, anionic and nonionic surfactants can be used. Preferred cationic species are quaternary ammonium salts, quaternary phosphonium salts and crown ethers. Examples of quaternary ammonium salts are tetrabutyl ammonium bromide, tetrabutyl ammonium hydrogen sulfate, tributylmethyl ammonium chloride, benzyltrimethyl ammonium chloride, benzyltriethyl ammonium chloride, methyltricaprylyl ammonium chloride, dodecyltrimethyl ammonium bromide, tetraoctyl ammonium bromide, cetyltrimethyl ammonium chloride, and trimethyloctadecyl ammonium hydroxide. Quaternary ammonium halides are useful in many systems, and the most preferred are dodecyltrimethyl ammonium bromide and tetraoctyl ammonium bromide.

The preferred surface active agents are those that will promote the formation of an emulsion between the organic and aqueous phases upon passing the liquids through a common mixing pump, but that will spontaneously separate the product mixture into aqueous and organic phases suitable for immediate separation by decantation or other simple phase separation procedures. One class of surface active agents that will accomplish this is liquid aliphatic C₁₅-C₂₀ hydrocarbons and mixtures of such hydrocarbons, preferably those having a specific gravity of at least about 0.82, and most preferably at least about 0.85. Examples of hydrocarbon mixtures that meet this description and are particularly convenient for use and readily available are mineral oils, preferably heavy or extra heavy mineral oil. The terms “mineral oil,” “heavy mineral oil,” and “extra heavy mineral oil” are well known in the art and are used herein in the same manner as they are commonly used in the art. Such oils are readily available from commercial chemicals suppliers throughout the world.

When an added emulsifying agent is used in the practice of this invention, the appropriate amount of agent to use is any amount that will perform as described above. The amount is otherwise not critical and may vary depending on the choice of the agent, and in the case of mineral oil, the grade of mineral oil. The amount may also vary with the fuel composition, the relative amounts of aqueous and organic phases, and the operating conditions. Appropriate selection will be a matter of routine choice and adjustment to the skilled engineer. In the case of mineral oil, best and most efficient results will generally be obtained using a volume ratio of mineral oil to the organic phase 1 of from about 0.00003 to about 0.003.

In certain embodiments of the invention, a metallic catalyst is included in the reaction system to regulate the activity of the hydroxyl radical produced by the hydroperoxide. Examples of such catalysts are transition metal catalysts, and preferably metals having atomic numbers of 21 through 29, 39 through 47, and 57 through 79. Particularly preferred metals from this group are nickel, sulfur, tungsten (and tungstates), cobalt, molybdenum, and combinations thereof. In certain systems within the scope of this invention, Fenton catalysts (ferrous salts) and metal ion catalysts in general such as iron (II), iron (III), copper (I), copper (II), chromium (III), chromium (VI), molybdenum, tungsten, cobalt, and vanadium ions, are useful. Of these, iron (II), iron (III), copper (II), and tungsten catalysts are preferred. For some systems, such as crude oil, Fenton-type catalysts are preferred, while for others, such as diesel-containing systems, tungsten or tungstates are preferred. Tungstates include tungstic acid, substituted tungstic acids such as phosphotungstic acid, and metal tungstates. In certain embodiments of the invention, nickel, silver, or tungsten, or combinations of these three metals, are particularly useful. The metallic catalyst when present will be used in a catalytically effective amount, which means any amount that will enhance the progress of the reaction (i.e., increase the reaction rate) toward the desired goal, particularly the oxidation of the sulfides to sulfones. The catalyst may be present as metal particles, pellets, flakes, shavings, or other similar forms, retained in the ultrasound chamber by physical barriers such as screens or other restraining means as the reaction medium is allowed to pass through.

The temperature of the combined aqueous and organic phases during ultrasound exposure may vary widely, although in most cases it is contemplated that the temperature will be within the range of from about 0° C. to about 500° C., preferably from about 20° C. to about 200° C., and most preferably from about 40° C. to about 125° C. In many cases, it will be beneficial to preheat the two phases, either individually or together, prior to their entry into the ultrasound chamber. The optimal degree of preheating will vary with the particular organic liquid to be treated and the ratio of aqueous to organic phases, provided that the temperature is not high enough to volatilize the organic liquid. With diesel fuel, for example, best results will most often be obtained by preheating the fuel to a temperature of at least about 70° C., and preferably from about 70° C. to about 100°. The aqueous phase may be preheated to any temperature up to its boiling point.

Ultrasound used in accordance with this invention consists of soundlike waves whose frequency is within or above the range of normal human hearing, i.e., in the range of 2 kHz (2,000 cycles per second) or above. Ultrasonic energy with frequencies as high as 10 gigahertz (10,000,000,000 cycles per second) has been generated, but for the purposes of this invention, useful results will be achieved with frequencies within the range of from about 2 kHz to about 100 kHz, and preferably within the range of from about 10 kHz to about 50 kHz. Ultrasonic waves can be generated from mechanical, electrical, electromagnetic, or thermal energy sources. The intensity of the sonic energy may also vary widely. For the purposes of this invention, best results will generally be achieved with an intensity ranging from about 10 watts/cm² to about 3000 watts/cm², or preferably from about 50 watts/cm² to about 1500 watts/cm². The typical electromagnetic source is a magnetostrictive transducer which converts magnetic energy into ultrasonic energy by applying a strong alternating magnetic field to certain metals, alloys and ferrites. The typical electrical source is a piezoelectric transducer, which uses natural or synthetic single crystals (such as quartz) or ceramics (such a barium titanate or lead zirconate) and applies an alternating electrical voltage across opposite faces of the crystal or ceramic to cause an alternating expansion and contraction of crystal or ceramic at the impressed frequency. Ultrasound has wide applications in such areas as cleaning for the electronics, automotive, aircraft, and precision instruments industries, flow metering for closed systems such as coolants in nuclear power plants or for blood flow in the vascular system, materials testing, machining, soldering and welding, electronics, agriculture, oceanography, and medical imaging. The various methods of producing and applying ultrasonic energy, and commercial suppliers of ultrasound equipment, are well known among those skilled in the use of ultrasound.

The exposure time of the multiphase reaction medium to ultrasound is not critical to the practice or to the success of the invention, and the optimal exposure time will vary according to the type of fuel being treated. An advantage of the invention however is that effective and useful results can be achieved with a relatively short exposure time. A preferred range of exposure times is from about 1 second to about 30 minutes, and a more preferred range is from about 15 seconds to about 1 minute.

Improvements in the efficiency and effectiveness of the process can also be achieved by recycling or secondary ultrasound treatments. A fresh supply of water may for example be added to the treated and separated organic phase to form a fresh emulsion which is then exposed to further ultrasound treatment, either on a batch or continuous basis. This re-exposure to ultrasound can be repeated multiple times for even better results. This is readily achieved in a continuous process by a recycle stream or by the use of a second stage ultrasound treatment, and possibly a third stage ultrasound treatment, with a fresh supply of water at each stage.

In systems where the reaction induced by ultrasound produces undesirable byproducts in the organic phase, these byproducts can be removed by conventional methods of extraction, absorption, or filtration. When the byproducts are polar compounds, for example, the extraction process can be any process that extracts polar compounds from a non-polar liquid medium. Such processes include solid-liquid extraction, using adsorbents such as silica gel, activated alumina, polymeric resins, and zeolites. Liquid-liquid extraction can also be used, with polar solvents such as dimethyl formamide, N-methylpyrrolidone, or acetonitrile. A variety of organic solvents that are either immiscible or marginally miscible with the fossil fuel, can be used. Toluene and similar solvents are examples.

The ultrasound-assisted reformation reaction generates heat, and with certain starting materials it is preferable to remove some of the generated heat to maintain control over the reaction. When gasoline is treated in accordance with this invention, for example, it is preferable to cool the reaction medium in the ultrasound chamber. Cooling is readily achievable by conventional means, such as the use of a liquid coolant jacket or a coolant circulating through a cooling coil in the interior of the ultrasound chamber. Water at atmospheric pressure is an effective coolant for these purposes. When cooling is achieved by immersing the ultrasound chamber in a coolant bath or by use of a circulating coolant, the coolant may be at a temperature of about 50° C. or less, preferably about 20° C. or less, and more preferably within the range of from about −5° C. to about 20° C. Suitable cooling methods or devices will be readily apparent to those skilled in the art. Cooling is generally unnecessary with diesel fuel, gas oils, and resids.

Operating conditions in general for the practice of this invention can vary widely, depending on the organic material being treated and the manner of treatment. The pH of the emulsion, for example, may range from as low as 1 to as high as 10, although best results are presently believed to be achieved within a pH range of 2 to 7. The pressure of the emulsion as it enters the ultrasound chamber can likewise vary, ranging from subatmospheric (as low as 5 psia or 0.34 atmosphere) to as high as 3,000 psia (214 atmospheres), although preferably less than about 400 psia (27 atmopheres), and more preferably less than about 50 psia (3.4 atmospheres), and most preferably from about atmospheric pressure to about 50 psia.

The operating conditions described in the preceding paragraphs that relate to ultrasound conditions, the inclusion of emulsion stabilizers and catalysts, and the general conditions of temperature and pressure apply to the process of the invention regardless of whether or not hydrogen peroxide or any other hydroperoxide is present in the reaction mixture. One of the unique and surprising discoveries of this invention is that the levels of sulfur-containing compounds and nitrogen-containing compounds are reduced substantially regardless of whether a hydroperoxide is present.

The process can be performed either in a batchwise manner or in a continuous-flow operation. Continuous-flow operations are preferred. In a currently preferred system, the ultrasound exposure is performed in a horizontal pipe reactor, 12 inches (30.5 cm) in diameter and 6 feet (1.83 m) in length, although a useful range of dimensions may be a diameter of from 4 inches to 24 inches (10.2 to 61 cm) and a length of 1 foot to 50 feet (30.5 to 1,524 cm), preferably from 6 feet to 12 feet (183 to 366 cm). The pipe is divided longitudinally into 5 sections or cells with perforated vertical walls separating the cells. A horizontal screen in each cell supports the metal catalyst particles and the perforated vertical walls serve to retain the particles in each cell. Ultrasound probes penetrate the top of the pipe and extend into the pipe interior, with one probe extending into each cell. Emulsion is passed through the pipe and thus through each cell in succession, at a rate of approximately 75 gallons/minute (4.7 liters per second, or 2,570 bbl/day). The volume ratio of organic to aqueous phases is 1:0.5.

The following examples are offered for purposes of illustration and are not intended to limit the scope of the invention.

### EXAMPLE 1

A petroleum-derived fuel was treated in accordance with the present invention in a stainless steel ultrasound chamber having an internal volume of 3 liters. A metal screen inside the chamber supported a bed of solid metal catalyst consisting of a mixture of tungsten flakes and nickel pellets, and positioned above the catalyst bed was an ultrasound probe whose lower end terminated approximately 5 cm above the catalyst bed. Ultrasound was supplied to the probe by an ultrasound generator as follows:

Ultrasound generator:

Supplier: Sonics & Materials, Inc., Newtown, Conn., USA

Power supply: net power output of 800 watts (run at 50%)

Voltage: 120 V, single phase

Current: 10 amps

Frequency: 20 kHz

The fuel used in this test was a 70/30 (volume ratio) mixture of straight-run diesel and FCC light cycle oil. Each fuel was combined with water at a ratio of approximately 2 parts by volume of fuel to 1 part by volume of water. Hydrogen peroxide was added as a 3% aqueous solution, at 0.0025 parts by volume of the solution to 1 part by volume of the water. The fuel and water were both preheated to 75° C. Extra heavy mineral oil, obtained from Mallinckrodt Baker Inc., Philipsburg, N.J., USA, was used as a surface active agent and added at a rate of approximately 1 drop of a 10% solution per 0.375 L of the total mixture. The mixture was heated to approximately 85° C. and emulsified with a high-speed shear mixer. The resulting emulsion was passed through the ultrasound chamber as a continuous stream at a flow rate of approximately 1 gallon per minute (3.8 liters/min). Residence time in the ultrasound chamber was approximately 1 minute.

The two-phase aqueous-organic mixture emerging from the ultrasound chamber passed through two cloth filters into a separation chamber. The organic phase was drawn from the top of the chamber and the aqueous phase from the bottom. The organic phase emerging from a single pass through the ultrasound chamber and separator was washed with acetonitrile. In some cases, the organic phase was recycled through the ultrasound chamber with fresh water at the same ratios and conditions used in the first pass. When recycling was performed twice, the sulfur content of the organic phase with a solvent wash was substantially the same as that achieved with a single pass followed by the solvent wash.

The fuel, both before and after treatment in the ultrasound chamber, was analyzed for concentrations of individual sulfur-containing compounds (test method: ASTM D-5623) and for total sulfur (test method: ASTM D-2622, using a sulfur analyzer Model SLFA-20, Horiba Instruments, Inc., Knoxville, Tenn., USA, with a detection limit of 20 ppm). The results, which demonstrate a sharp drop in the sulfur content, particularly among the thiophenes, are shown in Table II.

TABLE II

| Sulfur Components | | After Treatment |
|---|---|---|
| (ppm S by weight): | Before Treatment | and Solvent Extraction |
| benzothiophene | 10 | <1 |
| methyl benzothiophenes | 186 | <1 |
| dimethyl benzothiophenes | 486 | 1 |
| trimethyl benzothiophenes | 490 | 3 |
| tetramethyl benzothiophenes | 464 | 14 |
| dibenzothiophene | 169 | 2 |
| methyl dibenzothiophenes | 395 | 10 |
| dimethyl dibenzothiophenes | 450 | 12 |
| trimethyl dibenzothiophenes | 215 | 9 |
| unidentified volatile sulfur | 396 | 18 |
| hydrogen sulfide | <1 | <1 |
| sulfur dioxide | <1 | <1 |
| carbonyl sulfide | <1 | <1 |
| ethyl mercaptan | <1 | <1 |
| methyl sulfide | <1 | <1 |
| carbonyl disulfide | <1 | <1 |
| isopropyl mercaptan | <1 | <1 |
| ethylene sulfide | <1 | <1 |
| t-butyl mercaptan | <1 | <1 |
| n-propyl mercaptan | <1 | <1 |
| ethyl methyl sulfide | <1 | <1 |
| thiophene | <1 | <1 |
| sec-butyl mercaptan | <1 | <1 |
| isobutyl mercaptan | <1 | <1 |
| ethyl sulfide | <1 | <1 |
| n-butyl mercaptan | <1 | <1 |
| methyl disulfide | <1 | <1 |
| 2-methyl thiophene | <1 | <1 |
| 3-methyl thiophene | <1 | <1 |
| tetrahydrothiophene | <1 | <1 |
| ethyl methyl disulfide | <1 | <1 |
| 2-ethyl thiophene | <1 | <1 |
| 2,5-dimethyl thiophene | <1 | <1 |
| 3-ethyl thiophene | <1 | <1 |
| 2,4 & 2,3-dimethyl thiophene | <1 | <1 |
| 3,4-dimethyl thiophene | <1 | <1 |
| methyl ethyl thiophenes | <1 | <1 |
| trimethyl thiophenes | <1 | <1 |
| tetramethyl thiophenes | <1 | <1 |
| Total Sulfur by X-Ray | 0.3260 | 0.007 |
| Spectroscopy (weight %) | | |

The fuel was also given a full chemical analysis by ASTM D-5134 Mod. both before and after treatment, with results shown in Table III.

TABLE III

| | Before | |
|---|---|---|
| | Treatment | After Treatment |
| | (weight %) | (weight %) |
| n-hexane | 0.01 | |
| cyclohexane | 0.01 | |
| trans-1,2-dimethylcyclopentane | 0.01 | |
| n-heptane | 0.02 | |
| methylcyclohexane | 0.03 | |
| trans,cis-1,2,3-trimethylcyclopentane | 0.01 | |
| N,N-dimethyl formamide | | 0.30 |
| toluene | 0.01 | 0.19 |
| 2-methylheptane | 0.01 | |
| 3-methylheptane | 0.02 | 0.01 |
| trans-1,2-dimethylcyclohexane | 0.01 | 0.01 |
| n-octane | 0.06 | 0.03 |
| cis-1,2-dimethylcyclohexane | | 0.01 |
| 2,4-dimethylheptane | | 0.01 |
| ethylcyclohexane | 0.02 | 0.06 |
| 2,6-dimethylheptane | 0.01 | 0.01 |
| 1,1,3-trimethylcyclohexane | 0.01 | 0.06 |
| 2,5-dimethylheptane | | 0.01 |
| 3,5-dimethylheptane | | 0.01 |
| ethylbenzene | 0.01 | 0.02 |
| trans,trans-1,2,4-trimethylcyclohexane | 0.01 | 0.05 |
| meta-xylene | 0.02 | |
| para-xylene | 0.01 | |
| 2,3-dimethylheptane | | 0.02 |
| 2,3-dimethylheptane D/L | | 0.02 |
| 3-ethylheptane | | 0.01 |
| 4-methyloctane | 0.01 | 0.04 |
| 2-methyloctane | 0.01 | 0.05 |
| 3-methyloctane | 0.01 | |
| cis,cis-1,2,3-trimethylcyclohexane | | 0.01 |
| 3-ethylheptane | | 0.03 |
| 3-methyloctane | | 0.08 |
| cis,cis-1,2,4-trimethylcyclohexane | | 0.01 |
| orthoxylene | 0.02 | 0.01 |
| cis-1-ethyl-3-methylcyclohexane | 0.01 | 0.04 |
| trans-1-ethyl-4-methylcyclohexane | 0.01 | 0.09 |
| isobutylcyclopentane | 0.01 | 0.05 |
| 1-ethyl-1-methylcyclohexane | | 0.01 |
| cis,trans-1,2,3-trimethylcyclohexane | | 0.01 |
| trans,trans-1,2,3-trimethylcyclohexane | | 0.01 |
| n-nonane | 0.12 | 0.48 |
| trans-1-ethyl-3-methylcyclohexane | 0.01 | 0.07 |
| trans-1-ethyl-2-methylcyclohexane | | 0.02 |
| C₉ naphthenes | | 0.35 |
| 2,2-dimethyloctane | | 0.07 |
| isopropylcyclohexane | | 0.04 |
| cis-1-ethyl-2-methylcyclohexane | | 0.02 |
| sec-butylcyclopentane | 0.01 | |
| 2,4-dimethyloctane | | 0.05 |
| 2,6-dimethyloctane | 0.02 | 0.18 |
| 2,5-dimethyloctane | | 0.03 |
| n-propylcyclohexane | 0.02 | 0.17 |
| n-butylcyclopentane | 0.01 | 0.05 |
| 3,5-dimethyloctane | | 0.01 |
| n-propylbenzene | 0.02 | 0.04 |
| 3,6-dimethyloctane | 0.02 | 0.10 |
| 1-methyl-3-ethylbenzene | 0.02 | 0.04 |
| 1-methyl-4-ethylbenzene | 0.01 | |
| 2,3-dimethyloctane | 0.01 | 0.08 |
| 1,3,5-trimethylbenzene | 0.02 | 0.06 |
| 4-ethyloctane | | 0.07 |
| 5-methylnonane | 0.01 | 0.05 |
| 4-methylnonane | 0.01 | 0.13 |
| 1-methyl-2-ethylbenzene | 0.03 | 0.07 |
| 2-methylnonane | | 0.05 |
| 3-ethyloctane | 0.01 | 0.05 |
| 3-methylnonane | 0.02 | 0.17 |
| 1,2,3,5-tetramethylcyclohexane | | 0.01 |
| 1,2,3,4-tetramethylcyclohexane | 0.01 | 0.04 |
| 1,4-dimethyl-2-ethylcyclohexane | | 0.01 |
| 1,2,4-trimethylbenzene | 0.06 | 0.03 |
| cis-1-methyl-3-propylcyclohexane | 0.02 | 0.08 |
| cis-1,3-diethylcyclohexane | 0.01 | 0.12 |
| trans-1-methyl-3-propylcyclohexane | | 0.02 |
| trans-1,3-diethylcyclohexane | 0.01 | |
| 1-ethyl-2,3-dimethylcyclohexane | 0.01 | 0.04 |
| isobutylbenzene | 0.01 | 0.04 |
| cis-1-methyl-4-propylcyclohexane | | 0.02 |
| sec-butylbenzene | | 0.01 |
| n-decane | 0.03 | 0.75 |
| 1,3,5-trimethylbenzene | 0.01 | |
| 1,2,3,4-tetramethylcyclohexane | | 0.02 |
| trans-1,3-diethylcyclohexane | | 0.02 |
| 1,2,3-trimethylbenzene | | 0.01 |
| 1-methyl-3-isopropylbenzene | 0.01 | 0.02 |
| 1-methyl-4-isopropylbenzene | | 0.02 |
| 1-methyl-2-propylbenzene | | 0.01 |
| 2,3-dihydroindene | 0.01 | 0.03 |
| sec-butylcyclohexane | 0.04 | 0.04 |
| 1-methyl-2-isopropylbenzene | | 0.03 |
| butylcyclohexane | 0.01 | 0.12 |
| 1,3-diethylbenzene | 0.01 | 0.04 |
| trans-1-methyl-4-t-butylcyclohexane | 0.01 | 0.08 |
| 1-methyl-3-n-propylbenzene | 0.02 | 0.01 |
| 1-methyl-4-n-propylbenzene | 0.01 | |
| 1,4-diethylbenzene | 0.01 | |
| n-butylbenzene | 0.02 | |
| 1,3-dimethyl-5-ethylbenzene | 0.01 | 0.03 |
| 1,2-diethylbenzene | | 0.02 |
| 1-methyl-2-n-propylbenzene | 0.02 | 0.02 |
| cis-1-methyl-4-t-butylcyclohexane | 0.06 | 0.06 |
| 5-methyldecane | 0.03 | 0.04 |
| 4-methyldecane | 0.02 | 0.09 |
| 2-methyldecane | 0.02 | |
| 1,4-dimethyl-2-ethylbenzene | 0.03 | 0.09 |
| 1,3-dimethyl-4-ethylbenzene | 0.03 | 0.01 |
| 3-methyldecane | 0.02 | 0.06 |
| 1,2-dimethyl-4-ethylbenzene | 0.02 | 0.01 |
| 1,3-dimethyl-2-ethylbenzene | 0.01 | 0.02 |
| tricyclodecane | 0.03 | 0.04 |
| 1-methylindan | 0.01 | |
| 1,2-dimethyl-3-ethylbenzene | 0.03 | 0.02 |
| n-undecane | 0.63 | 0.39 |
| 1,2,4,5-tetramethylbenzene | 0.02 | 0.01 |
| 1,2,3,5-tetramethylbenzene | 0.05 | 0.03 |
| C₁₁ unidentified | 0.10 | 0.39 |
| 4-methylindan | 0.04 | 0.02 |
| 5-methylindan | 0.03 | 0.02 |
| 1,2,3,4-tetramethylbenzene | 0.07 | 0.03 |
| 2-methylindan | 0.02 | |
| tetralin | 0.01 | 0.01 |
| 6-methylundecane | 0.03 | 0.05 |
| 5-methylundecane | 0.02 | 0.06 |
| 4-methylundecane | 0.04 | 0.05 |
| 3-methylundecane | | 0.12 |
| 2-methylundecane | 0.05 | |
| naphthalene | 0.13 | |
| naphthalene3-methylundecane | 0.05 | |
| C₁₁ aromatics | 0.45 | 0.54 |
| n-dodecane | 0.91 | 0.40 |
| dodecanes | 0.50 | 0.58 |
| 2-methylnaphthalene | 1.19 | |
| 1-methylnaphthalene | 0.81 | |
| tridecanes | 3.30 | 2.55 |
| n-tridecane | 2.83 | 1.00 |
| tetradecanes | 6.95 | 5.01 |
| n-tetradecane | 4.47 | 1.63 |
| pentadecanes | 9.55 | 6.52 |
| n-pentadecanes | 5.30 | 2.46 |
| hexadecanes | 10.20 | 7.20 |
| n-hexadecane | 3.57 | 2.43 |
| heptadecanes | 10.61 | 8.44 |
| n-heptadecanes | 3.60 | 3.13 |
| pristane | 1.28 | 1.78 |
| octadecanes | 7.52 | 7.46 |
| n-octadecane | 1.97 | 2.71 |
| phytane | 0.83 | 1.46 |
| nonadecanes | 5.85 | 7.20 |
| n-nonadecane | 1.77 | 2.79 |
| eicosanes | 5.63 | 7.14 |
| n-eicosane | 0.97 | 2.71 |
| heneicosanes | 4.30 | 7.35 |
| docosanes | 2.14 | 5.74 |
| tricosanes | 0.50 | 3.30 |
| tetracosanes | 0.04 | 1.18 |
| pentacosanes | 0.01 | 0.10 |
| unidentified | | 0.16 |
| Total: | 100.00 | 100.00 |
| Molecular Weight of Sample | 220.24 | 229.13 |
| Molecular Weight of C₆ Plus | 220.24 | 230.24 |
| Density of Sample | 0.8245 | 0.8680 |
| Density of C₆ Plus | 0.8245 | 0.8280 |

### EXAMPLE 2

Using the same equipment and procedures as Example 1, four fuels were treated in accordance with the invention. The fuels were straight-run diesel, feed-rack diesel, light cycle oil, and a 70/30 (by volume) blend of straight-run diesel and light cycle oil. Various properties and analytical data, obtained by conventional methods widely used in the industry, are listed in Table IV below.

TABLE IV
Comparisons Before (B) and After (A) Treatment for Four Different Fuels
(Treatment Includes Solvent Extraction)

| | | | | | | 70/30 Vol. | | | |
|---|---|---|---|---|---|---|---|---|---|
| | | | | | | Ratio | | | |
| | (Indus- | | | | | Straight Run | | | |
| | try | Straight-Run | | Feed Rack | | Diesel/Light | | | |
| | Specifi- | Diesel | | Diesel | | Cycle Oil | | Light Cycle Oil | |
| | cation) | B | A | B | A | B | A | B | A |
| Gravity, API | 30-40 | 32.8 | 40.5 | 33.9 | 38.2 | 33.0 | 38.2 | 21.4 | 42.6 |
| Total sulfur, | 15 | 2,500 | 5 | 228 | 64 | 228 | 64 | 5,572 | 8 |
| ppm (wt) | | | | | | | | | |
| Cetane Index | 41 | 46.5 | 63 | 46 | 55 | 46 | 55 | 30.0 | 64.5 |
| Copper strip | | | | | | | | 1A | 1A |
| Flash | | | | 160 | 172 | | | 230 | 124 |
| Ramsbottom | 0.35 | | 0.15 | 0.29 | 0.07 | 0.29 | 0.07 | 0.38 | 0.05 |
| Carbon | | | | | | | | | |
| Viscosity | | | 4 | 2.9 | 3.4 | 2.9 | 3.4 | 3.5 | 3.7 |
| Middle Distillate | | | | | | | | | |
| Tests: | | | | | | | | | |
| Pour Point | 10 | 0 | 25 | −15 | 0 | 5 | 25 | 10 | 25 |
| (° F., Summer) | | | | | | | | | |
| Cloud Point | 36 | 16 | 36 | −4 | 12 | 8 | 34 | 20 | 34 |
| (° F., Summer) | | | | | | | | | |
| Color | | | <0.5 | | 0.5 | | 0.5 | 1.5 | <0.5 |
| Flash | 125 | 154 | 164 | 160 | 172 | 196 | 180 | | |

Distillation, D-86 ° F.

| Initial Boiling Point | | | 486 | 426 |
|---|---|---|---|---|
| 5% | | | 488 | 493 |
| 10% | | | 497 | 505 |
| 20% | | | 509 | 517 |
| 30% | | | 521 | 528 |
| 40% | | | 534 | 540 |
| 50% | | | 549 | 554 |
| 60% | | | 566 | 568 |
| 70% | | | 586 | 584 |
| 80% | | | 608 | 601 |
| 90% | | | 631 | 625 |
| 95% | | | 654 | 644 |
| End Point | | | 667 | 653 |
| Recovery | | | 96.5 | 96.5 |
| Residual | | | 1.1 | 1.8 |
| Loss | | | 2.4 | 1.7 |
| Paraffins | 48.8 | 62.8 | 32.5 | 36.1 |
| Monocyclo- | 14.8 | 18.3 | 8.5 | 12.6 |
| paraffins | | | | |
| Dicycloparaffins | 5.3 | 8.1 | 3.6 | 8.2 |
| Tricycloparaffins | 0.9 | 1.6 | 0.8 | 1.5 |
| Paraffins Subtotal | 69.8 | 90.8 | 45.4 | 58.4 |
| Naphthalenes | 9.0 | 0.5 | 20.3 | 12.1 |
| Acenaphthenes | 3.0 | 0.2 | 7.6 | 4.7 |
| Acenaphthalenes | 1.5 | 0.2 | 4.2 | 2.6 |
| Naphthalenes Subtotal | 13.5 | 0.9 | 32.1 | 19.4 |
| Alkylbenzenes | 6.0 | 3.0 | 5.7 | 6.2 |
| Indanes/Tetralins | 6.3 | 3.5 | 7.9 | 9 |
| Indenes | 2.0 | 1.7 | 2.1 | 3.1 |
| Tricyclic Aromatics | 2.4 | 0.1 | 6.8 | 3.9 |
| Aromatics Subtotal | 16.7 | 8.3 | 22.5 | 22.2 |
| Total | 100.0 | 100.0 | 100.0 | 100.0 |

The last 15 rows of Table IV demonstrate the effect of the invention in converting olefins to paraffins, saturating aromatics and opening rings. In particular, the increase in cycloparaffins shows saturation of aromatic rings, the lowering of the level of naphthalenes and the accompanying increase in indanes and tetralins demonstrate at least partial saturation of aromatics, the drop in acenaphthalenes and acenaphthenes and the rise in indanes and indenes demonstrates at least partial saturation and ring opening, the lowering of tricyclic aromatics (such as anthracenes and phenanthrenes) demonstrates at least partial saturation and ring opening, and the increase in alkybenzenes demonstrates ring opening. The “Total Sulfur” and “Cetane Index” data demonstrate the large reduction in sulfur level and the large improvement in cetane index.

### EXAMPLE 3

A reaction vessel was charged with 250 mL FCC light cycle oil, 125 mL of water, hydrogen peroxide at a concentration of 25 ppm to a pH of 2-4, one drop of a mixture of 90 parts straight-run diesel and 10 parts of extra heavy mineral oil, and a mixture of tungsten flakes (17 g) and nickel pellets (17 g). The resulting mixture was heated to 180° F. (82° C.), then emulsified with a high-speed shear mixer at 2000 rpm for 5 seconds. An ultrasound probe, rated for 20 kHz and driven by a 750 W generator, was then placed in the solution and energized, operating at 40% intensity. After 30 seconds, the ultrasound probe was removed. The aqueous-organic mixture was then placed in a separatory funnel and allowed to separate for 5 minutes. Analytical results of the organic phase are shown in Table V:

TABLE V
Comparisons Before and After Treatment for Light Cycle Oil

| | Before | After |
|---|---|---|
| | Ultrasound | Ultrasound |
| Gravity, API | 22.6 | 27.3 |
| Cetane Index | 34.5 | 39.1 |
| Total Sulfur, ppm | 7073 | 3917 |
| Total Nitrogen, ppm | 721 | 415 |
| Carbon, weight % (ASTM | 88.24 | 87.01 |
| D-5291) | | |
| Hydrogen, weight % (ASTM | 10.57 | 12.05 |
| D-5291) | | |
| Paraffins and Aromatics | | |
| (ASTM D-2549): | | |
| Acyclic Paraffins | 32.1 | 35.8 |
| Monocycloparaffins | 9.8 | 14.0 |
| Dicycloparaffins | 4.3 | 9.2 |
| Tricycloparaffins | 0.8 | 1.6 |
| Paraffins Subtotal | 46.2 | 60.6 |
| Naphthalenes | 16.1 | 9.4 |
| Acenaphthenes | 6.8 | 3.9 |
| Acenaphthalenes | 3.6 | 2.2 |
| Alkylbenzenes | 7.5 | 7.1 |
| Indanes/Tetralins/Indenes | 12.6 | 13.5 |
| Tricyclic Aromatics | 6.4 | 3.3 |
| Aromatics Subtotal | 53.0 | 39.4 |

Like Table IV, Table V demonstrates the effect of the invention in converting olefins to paraffins, saturating aromatics and opening rings. The increase in cycloparaffins shows saturation of aromatic rings, the reduction in naphthalenes and the accompanying increase in indanes and tetralins demonstrate at least partial saturation of aromatics, the reduction in acenaphthalenes and acenaphthenes and the increase in indanes and indenes demonstrates at least partial saturation and ring opening, the reduction in tricyclic aromatics (such as anthracenes and phenanthrenes) demonstrates at least partial saturation and ring opening, and the increase in alkybenzenes demonstrates ring opening. Also demonstrated are a reduction in nitrogen-bearing compounds and sulfur-bearing compounds, plus increases in API gravity, cetane index and hydrogen content.

### EXAMPLE 4

A mixture was prepared by combining 5 gallons (19 L) of FCC light cycle oil, 2.5 gallons (9.5 L) of water containing 2 mL of heavy mineral oil, and hydrogen peroxide at a concentration of 25 ppm to a pH of 2-4. The mixture was passed through the continuous-flow ultrasound reactor of Example 1 under the conditions described in that example with a liquid hourly space velocity (LHSV) of 30 seconds. The separated organic phase was then desulfurized in a standard industry hydrodesulfurization (HDS) unit at the following conditions: temperature 650° F. (343° C.), pressure 500 psi (35 atmospheres), hydrogen circulation rate 1,000 SCF/bbl (standard cubic feet per barrel), and LHSV=2. The API gravity, cetane index, total nitrogen, and total sulfur were determined for the initial untreated light cycle oil, the light cycle oil after ultrasound treatment, the light cycle oil after hydrodesulfurization but without ultrasound, and the light cycle oil having both been treated with ultrasound and undergone hydrodesulfurization. The results are listed in Table VI:

TABLE VI
Comparisons Before and After Treatment for Light Cycle Oil

| | Before HDS | | After HDS | |
|---|---|---|---|---|
| | Without | With | Without | With |
| | Ultrasound | Ultrasound | Ultrasound | Ultrasound |
| Gravity, API | 22.6 | 33.8 | 26.9 | 34.0 |
| Cetane Index | 34.5 | 47.5 | 39.7 | 47.6 |
| Nitrogen, ppm | 721 | 170 | 51.5 | 86 |
| Sulfur, ppm | 7073 | 1121 | 557 | 80 |
| Hydrogen | | | 450 | 100 |
| usage SCF/bbl | | | | |

The data in Table VI indicate that the ultrasound treatment increases both API gravity and cetane index, regardless of whether hydrodesulfurization has been performed, and decreases both sulfur and nitrogen in addition to the decreases in these elements that is achieved by hydrodesulfurization. Note also the reduction in hydrogen usage due to ultrasound.

### EXAMPLE 5

A reaction mixture not containing hydrogen peroxide or any other hydroperoxide was prepared by combining:

250 mL of a mixture of 50% FCC slurry oil and 50% 1000-psi-hydrotreated kerosene,

125 mL of water, and

one drop of a mixture of 90 parts diesel and 10 parts heavy mineral oil surfactant (by volume).

The reaction mixture was heated to 180° F. (82° C.), then emulsified with a high-speed shear mixer at 2000 rpm for 5 seconds. An ultrasound probe was then placed in the reaction mixture, energized for 60 seconds and removed. The reaction mixture was then allowed to separated for ten minutes in a separatory funnel. The aqueous phase was removed from the bottom of the funnel and the oil phase was recovered and retreated three times with ultrasound in the same manner, each treatment followed by phase separation and combining of the oil phase with fresh water. The laboratory analyses of the starting material and the fourth-run product (exclusive of the kerosene) are listed in Table VII.

TABLE VII
Comparisons Before and After Treatment for FCC Slurry Oil

| | Starting | 4th Run |
|---|---|---|
| | Material | Product |
| Gravity, API | 4.8 | 31.2 |
| Total Nitrogen, ppm | 2,048 | 500 |
| Total Sulfur, ppm | 11,380 | 2,252 |
| Bromine Number | 14 | 2 |
| Paraffins and Aromatics (ASTM D-2549): | | |
| Acyclic Paraffins | 9.4 | 45.8 |
| Monocycloparaffins | 9.9 | 16.9 |
| Dicycloparaffins | 7 | 10.2 |
| Tricycloparaffins | 1.8 | 1.4 |
| Alkylbenzenes | 15.3 | 7.5 |
| Indanes/Tetralins/Indenes | 21.4 | 11.8 |
| Naphthalenes | 16.2 | 2.6 |
| Acenaphthenes | 8 | 1 |
| Acenaphthalenes | 4.4 | 0.8 |
| Tricyclic Aromatics | 15.2 | 2 |
| Distillation D 2887 90% Point (° F.) | 900 | 739 |

Of particular significance in Table VII are the reduction in sulfur and nitrogen levels, the increase in cycloparaffins resulting from saturation of aromatic rings, the reduction in the distillation temperature of the 90% distillation point, and the increase in API gravity.

### EXAMPLE 6

A reaction mixture not containing hydrogen peroxide or any other hydroperoxide was prepared by combining:

250 mL of a mixture of 50% coker gas oil and 50% 1000-psi-hydrotreated kerosene,

125 mL of water, and

one drop of a mixture of 90 parts diesel and 10 parts heavy mineral oil surfactant (by volume).

The reaction mixture was heated to 180° F. (82° C.), then emulsified with a high-speed shear mixer at 2000 rpm for 5 seconds. An ultrasound probe was then placed in the reaction mixture, energized for 60 seconds and removed. The reaction mixture was then allowed to separated for ten minutes in a separatory funnel. The aqueous phase was removed from the bottom of the funnel and the oil phase was recovered and retreated three times with ultrasound in the same manner, each treatment followed by phase separation and combining of the oil phase with fresh water. The laboratory analyses of the starting material and the fourth-run product (exclusive of the kerosene) are listed in Table VIII.

TABLE VIII
Comparisons Before and After Treatment for Coker Gas Oil

| | Starting | 4th Run |
|---|---|---|
| | Material | Product |
| Gravity, API | 13.0 | 33.1 |
| Total Nitrogen, ppm | 3.514 | 976 |
| Total Sulfur, ppm | 36,920 | 10,860 |
| Bromine Number | 10 | 2 |
| Pour Point (° F.) | 97 | 10 |
| Paraffins and Aromatics (ASTM D-2549): | | |
| Acyclic Paraffins | 23.6 | 46.4 |
| Monocycloparaffins | 8.7 | 16.1 |
| Dicycloparaffins | 5.4 | 9.4 |
| Tricycloparaffins | 1.2 | 1.2 |
| Alkylbenzenes | 15.1 | 9.1 |
| Indanes/Tetralins/Indenes | 20.0 | 12.0 |
| Naphthalenes | 9.6 | 2.4 |
| Acenaphthenes | 4.4 | 0.8 |
| Acenaphthalenes | 4.2 | 1 |
| Tricyclic Aromatics | 7.8 | 1.6 |
| Distillation D 2887 90% Point (° F.) | 899 | 744 |

Of particular significance in Table VIII are the reduction in sulfur, nitrogen, bromine number (an indicator of the levels of olefins and diolefins), the sharp drop in pour point, the sharp increase in API gravity, and the increase in cycloparaffins resulting from saturation of aromatic rings.

### EXAMPLE 7

A reaction mixture not containing hydrogen peroxide or any other hydroperoxide was prepared by combining:

250 mL of a mixture of 50% coker distillate,

125 mL of water, and

one drop of a mixture of 90 parts diesel and 10 parts heavy mineral oil surfactant (by volume).

The reaction mixture was heated to 180° F. (82° C.), then emulsified with a high-speed shear mixer at 2000 rpm for 5 seconds. An ultrasound probe was then placed in the reaction mixture, energized for 30 seconds and removed. The reaction mixture was then allowed to separated for ten minutes in a separatory funnel. The aqueous phase was removed from the bottom of the funnel and the oil phase was recovered and retreated three times with ultrasound in the same manner, each treatment followed by phase separation and combining of the oil phase with fresh water. The laboratory analyses of the starting material and the fourth-run product (exclusive of the kerosene) are listed in Table IX.

TABLE IX
Comparisons Before and After Treatment for Coker Gas Oil

| | Starting | 4th Run |
|---|---|---|
| | Material | Product |
| Gravity, API | 30.0 | 32.8 |
| Cetane index | 42.8 | 44.7 |
| Total Sulfur, ppm | 26,900 | 8,960 |
| Total Nitrogen, ppm | 1,057 | 440 |
| Bromine Number | 28 | 9 |
| Pour Point (° F.) | 10 | −10 |
| Paraffins and Aromatics (ASTM D-2549): | | |
| Acyclic Paraffins | 36.4 | 38.5 |
| Monocycloparaffins | 13.0 | 16.1 |
| Dicycloparaffins | 9.4 | 12.6 |
| Tricycloparaffins | 1.9 | 2.2 |
| Alkylbenzenes | 11.9 | 8.5 |
| Indanes/Tetralins/Indenes | 12.7 | 13.7 |
| Naphthalenes | 6.6 | 4.1 |
| Acenaphthenes | 2.9 | 1.8 |
| Acenaphthalenes | 2.0 | 1.2 |
| Tricyclic Aromatics | 3.2 | 1.3 |

Of particular significance in Table IX are the reduction in sulfur, nitrogen, aromatics, and bromine number, and the increases in API gravity and cetane index.

The foregoing is offered primarily for purposes of illustration. Further variations in the materials, additives, operating conditions, and equipment that are still within the scope of the invention will be readily apparent to those skilled in the art.

We claim:

1. A process for treating a crude oil fraction to reduce levels therein of aromatics, olefins, sulfur-bearing compounds, and nitrogen-bearing compounds, and to raise the API gravity thereof, said process comprising:
(a) combining said crude oil fraction with an aqueous liquid to form an emulsion;
(b) exposing said emulsion to ultrasound;
(c) separating said emulsion after said ultrasound exposure into aqueous and organic phases; and
(d) recovering said organic phase from said aqueous phase.

2. A process in accordance with claim 1 in which no hydroperoxide is added to said crude oil fraction or incorporated into said aqueous liquid in forming said emulsion.

3. A process in accordance with claim 1 in which step (a) comprises incorporating a hydroperoxide into said emulsion.

4. A process in accordance with claim 1 in which said crude oil fraction is a fraction boiling within the diesel range.

5. A process in accordance with claim 4 in which said crude oil fraction is a member selected from the group consisting of fluid catalytic cracking (FCC) cycle oil fractions, coker distillate fractions, straight run diesel fractions, and blends thereof.

6. A process in accordance with claim 1 in which said crude oil fraction is a fraction boiling within the gas oil range.

7. A process in accordance with claim 6 in which said crude oil fraction is a member selected from the group consisting of FCC cycle oil, FCC slurry oil, light gas oil, heavy gas oil, and coker gas oil.

8. A process in accordance with claim 1 in which said crude oil fraction is a member selected from the group consisting of gasoline, jet fuel, straight-run diesel, blends of straight-run diesel and FCC light cycle oil, and petroleum residuum-based fuel oils.

9. A process in accordance with claim 1 in which said ultrasound exposure is at a sufficient intensity and time to cause an increase in API gravity by an amount ranging from 2 to 30 API units.

10. A process in accordance with claim 1 in which said ultrasound exposure is at a sufficient intensity and time to cause an increase in API gravity by an amount ranging from 7 to 25 API units.

11. A process in accordance with claim 4 in which said ultrasound exposure is at a sufficient intensity and time to raise the cetane index of said diesel range crude oil fraction by an amount ranging from 1 to 40 units.

12. A process in accordance with claim 4 in which said ultrasound exposure is at a sufficient intensity and time to raise the cetane index of said diesel range crude oil fraction by an amount ranging from 4 to 20 units.

13. A process in accordance with claim 1 in which step (b) is performed at an ultrasound intensity of from about 10 watts/cm² to about 3000 watts/cm².

14. A process in accordance with claim 1 in which step (b) is performed at an ultrasound intensity of from about 50 watts/cm² to about 1500 watts/cm².

15. A process in accordance with claim 1 in which step (b) is performed at an ultrasound exposure time of from about 1 second to about 30 minutes.

16. A process in accordance with claim 1 in which step (b) is performed at an ultrasound exposure time of from about 15 seconds to about 1 minute.

17. A process in accordance with claim 1 further comprising contacting said emulsion with a transition metal catalyst during step (b).

18. A process in accordance with claim 17 in which said transition metal catalyst is a member selected from the group consisting of metals having atomic numbers of 21 through 29, 39 through 47, and 57 through 79.

19. A process in accordance with claim 17 in which said transition metal catalyst is a member selected from the group consisting of nickel, silver, tungsten, cobalt, molybdenum, and combinations thereof.

20. A process in accordance with claim 17 in which said transition metal catalyst is a member selected from the group consisting of nickel, silver, tungsten, and combinations thereof.

21. A process in accordance with claim 1 in which step (a) comprises combining said crude oil fraction and said aqueous fluid at a (crude oil fraction):(aqueous fluid) volume ratio of from about 8:1 to about 1:5.

22. A process in accordance with claim 1 in which step (a) comprises combining said crude oil fraction and said aqueous fluid at a (crude oil fraction):(aqueous fluid) volume ratio of from about 5:1 to about 1:1.

23. A process in accordance with claim 1 in which step (a) comprises combining said crude oil fraction and said aqueous fluid at a (crude oil fraction):(aqueous fluid) volume ratio of from about 4:1 to about 2:1.

24. A process in accordance with claim 3 in which said hydroperoxide constitutes from about 10 ppm to about 100 ppm by weight of said aqueous liquid.

25. A process in accordance with claim 3 in which said hydroperoxide constitutes from about 15 ppm to about 50 ppm by weight of said aqueous liquid.

26. A process in accordance with claim 1 in which said hydroperoxide is hydrogen peroxide.

27. A process in accordance with claim 1 in which said aqueous liquid is an aqueous solution of hydrogen peroxide at a concentration of from about 15 ppm to about 50 ppm by weight.

28. A process in accordance with claim 1 further comprising preheating said crude oil fraction to a temperature of from about 20° C. to about 200° C. prior to step (b).

29. A process in accordance with claim 1 further comprising preheating said crude oil fraction to a temperature of from about 40° C. to about 125° C. prior to step (b).

30. A process in accordance with claim 1 in which step (b) is performed at a pressure of less than 400 psia.

31. A process in accordance with claim 1 in which step (b) is performed at a pressure of less than 50 psia.

32. A process in accordance with claim 1 in which step (b) is performed at a pressure within the range of from about atmospheric pressure to about 50 psia.

33. A process in accordance with claim 1 further comprising preheating said crude oil fraction to a temperature of from about 40° C. to about 125° C. prior to step (b).

34. A process in accordance with claim 1 further comprising incorporating an emulsion stabilizer in said emulsion.

35. A process in accordance with claim 34 in which said emulsion stabilizer is a mixture of aliphatic C₁₅-C₂₀ hydrocarbons having a specific gravity of at least about 0.82.

36. A process in accordance with claim 35 in which said mixture of aliphatic C₁₅-C₂₀ hydrocarbons has a specific gravity of at least about 0.85.

37. A process in accordance with claim 35 in which said mixture of aliphatic C₁₅-C₂₀ hydrocarbons is heavy mineral oil.

38. A process for treating an organic liquid comprising fused-ring aromatic compounds and olefinic compounds to lower the density of said organic liquid, said process comprising:
(a) combining said organic liquid with an aqueous liquid to form an emulsion;
(b) subjecting said emulsion to ultrasound at a sufficient intensity and for a sufficient time to open fused rings in fused-ring aromatic compounds in said organic liquid and to convert olefinic compounds in said organic liquid to paraffins;
(d) permitting said emulsion to separate into aqueous and organic phases of which said organic phase comprises said organic liquid; and
(e) isolating said organic liquid from said aqueous phase.

39. A process in accordance with claim 38 in which said aqueous liquid is an aqueous solution of a hydroperoxide at a concentration of from about 10 ppm to about 100 ppm by weight.

40. A process in accordance with claim 38 in which said aqueous liquid is an aqueous solution of hydrogen peroxide at a concentration of from about 15 ppm to about 50 ppm by weight.

41. A process in accordance with claim 38 further comprising contacting said emulsion with a transition metal catalyst while subjecting said emulsion to said ultrasound in step (b).

42. A process in accordance with claim 41 in which said transition metal catalyst is a member selected from the group consisting of nickel, silver, tungsten, and combinations thereof.

43. A process in accordance with claim 1 in which step (b) is performed in batchwise manner.

44. A process in accordance with claim 1 in which step (b) is performed in continuous-flow manner.

45. A process for removing organic sulfur from a crude oil fraction, said process comprising:
(a) combining said crude oil fraction with an aqueous liquid to form an emulsion;
(b) exposing said emulsion to ultrasound;
(c) separating said emulsion after said ultrasound exposure into aqueous and organic phases;
(d) recovering said organic phase from said aqueous phase; and
(e) contacting said organic phase with hydrogen gas under conditions causing conversion of said organic sulfur to sulfur dioxide by hydrodesulfurization.

46. A process in accordance with claim 45 in which no hydroperoxide is added to said crude oil fraction or incorporated into said aqueous liquid in forming said emulsion.

47. A process in accordance with claim 45 in which said crude oil fraction is a fraction boiling within the diesel range.

48. A process in accordance with claim 47 in which said crude oil fraction is a member selected from the group consisting of fluid catalytic cracking (FCC) cycle oil fractions, coker distillate fractions, straight run diesel fractions, and blends thereof.

49. A process in accordance with claim 45 in which said crude oil fraction is a fraction boiling within the gas oil range.

50. A process in accordance with claim 49 in which said crude oil fraction is a member selected from the group consisting of FCC cycle oil, FCC slurry oil, light gas oil, heavy gas oil, and coker gas oil.
